(19) United States
(12) Patent Application Publication
(10) Pub. No.: US 2005/0002943 A1
(43) Pub. Date: Jan. 6, 2005

(54) Amp-kinase agonists or adenosine pro-drugs as immuno-stimulating agents

(76) Inventors: Oberdan Leo, Wezembeek-Oppem (BE); Erika Baus, Wezembeek-Oppem (BE)

Correspondence Address: HOWREY SIMON ARNOLD & WHITE LLP, 750 BERING DRIVE, HOUSTON, TX, 77057, US

(21) Appl. No.: 10/494,344

(22) PCT Filed: Oct. 30, 2002
(86) PCT No.: PCT/EP02/12102

(30) Foreign Application Priority Data
Oct. 31, 2001 (EP) 01870233.2

(51) Int. Cl. A61K 39/38; A61K 39/00; A61K 31/7056

(52) U.S. Cl. 424/184.1; 514/43

(57) ABSTRACT

The present invention relates to the use of AMP-activated protein kinase (AMP-kinase) agonists or adenosine pro-drugs as immune enhancing compounds, as adjuvants in a vaccine or as anti-inflammatory compounds. The invention further relates to a compositions, vaccines and products comprising an immune response eliciting molecule and an immune response enhancing compound, wherein said immune enhancing compound is chosen from the group of AMP-activated protein kinase (AMP-kinase) agonists or adenosine pro-drugs.

### FIELD OF THE INVENTION

The present invention relates to immune response enhancing compounds and more specifically to AMP-kinase agonist or adenosine pro-drug for use in human and animals. The present invention further relates to uses of said compounds as adjuvants and to vaccines comprising said compounds.

### BACKGROUND TO THE INVENTION

Vaccines have traditionally consisted of live attenuated pathogens, inactivated organisms or inactivated toxins. Although these have proved successful in the past, several drawbacks have limited their use. New approaches to vaccine development have emerged in the past decades including recombinant protein subunits, synthetic peptides and plasmid DNA. These offer significant advantages over traditional approaches such as reduced toxicity. However, the new-generation vaccines are poorly immunogenic when administered lone, and therefore, a great need exists for immunological adjuvants.

An adjuvant is a substance added for example to a medicine or a vaccine to increase its effectiveness. When added to a vaccine said adjuvant enhances the immunogenicity of the antigen administered. Adjuvants may act through two basic mechanisms. The first mechanism is the ability of adjuvants to enhance long-term release of the antigen by functioning as a depot, increasing thus the length of time the immune system is presented with the antigen. The second mechanism is related to the capacity of adjuvants to directly stimulate or modulate the activity of immune cells. In particular, many adjuvants increase cellular infiltration and trafficking to the injection site, promote the activation state of antigen-presenting cells (APC) by up-regulating co-stimulatory signals or MHC (major histocompatibility complex) expression, and stimulate cytokine release. Consequently, these compounds often cause inflammation, tissue necrosis and eventually pain and/or discomfort.

Safety represents an important issue in adjuvant development. For standard prophylactic immunization in healthy individuals, only adjuvants that induce minimal side effects will prove acceptable. In contrast, for adjuvants that are designed to be used in life-threatening situations (e.g. cancer), the acceptable level of adverse events is likely to be higher. Many experimental adjuvants have demonstrated high potency in clinical trials but most have proved too toxic for routine clinical use. At present, the choice of adjuvants for both animal and human vaccination reflects a compromise between a requirement for adjuvanticity and an acceptable low level of side effects. To date, the only adjuvants licensed for human use are aluminum-based mineral salts (generically called alum), emulsions (MF59) and virosomes.

Several documents disclose the use of guanosine derivatives as adjuvant. U.S. Pat. No. 5,011,828 relates to guanine nucleoside derivatives that are substituted at the 7- and 8-positions of the guanine ring as immune response enhancing compounds. U.S. Pat. No. 4,539,205 describes modulation of animal cellular responses with 8-substituted guanine derivatives bonded 9-1′ to an aldose having 5 or 6 carbon atoms in the aldose chain. U.S. Pat. No. 4,643,992 discloses the use of derivatives of 8-hdroxy guanosine, 7-methyl-8-oxoguanosine and 7-methyl-8-thioxoguanosine in modulating animal cellular response. The guanosine analogues described above have the disadvantage of displaying mitogenic properties in vivo.

Arai et al. (2000: Gene therapy, vol 7, No 8, pages 694-702) describes a vaccine composition comprising DNA and 8-Br-cAMP, wherein 8-Br-cAMP is regarded as an immune enhancing molecule. The DNA vaccine consists of a HIV-1 Env sequence placed under the control of the CMV promoter. The CMV promoter contains a CAMP responsive element (CRE) and elevation in intracellular concentrations of cAMP results in the upregulation of CRE and enhancement of the transcriptional activity of the CMV promoter. It is described in Arai et al. that 8-Br-cAMP enhances CAMP and that the adjuvant effect of 8-Br-cAMP on the DNA vaccine occurs by enhancing the activity of the CMV promoter, which sequence is comprised in the DNA itself. The adjuvant effect of 8-Br-cAMP is thus dependent from the choice of the promoter in the DNA vaccine, and probably restricted to CMV-promoter/CRE elements.

It is one of the aims of the present invention to provide novel compounds for enhancing the immune response to a given antigen without apparent side effects. It is a further aim of the present invention to provide compounds for diminishing side effects of known adjuvants in pharmaceutical compositions. It is also an aim of the present invention to provide novel formulations for vaccine compositions.

### SUMMARY OF THE INVENTION

In a first aspect, the present invention provides means for enhancing the immune response to a given antigen by the co-administration of at least one AMP-kinase agonists and/or adenosine pro-drug with said antigen. More in particular, the present invention relates to a composition comprising an immune response eliciting compound and an immune response enhancing compound chosen from the group of the AMP-activated protein kinase (AMP-kinase) agonists or the adenosine pro-drugs. According to an embodiment the AMP-kinase agonist is an AMP (adenosine monophosphate) mimetic or pro-drug or derivative thereof which stimulates AMP-kinase. An example of AMP-kinase agonist and adenosine pro-drug compound displaying both actions is the small molecular weight (MW: 258.2) natural compound 5-aminoimidazole-4-carboxamide riboside (AICA-riboside or acadesine), which is known as an intermediate in the de novo purine biosynthesis. Another example of AMP-kinase agonist is 6-MPR (6-mercaptopurine riboside).

In a second aspect, the present invention relates to the use of an AMP-kinase agonist or an adenosine pro-drug as an immune response enhancing compound. The invention also relates to the use of said AMP-kinase agonist or adenosine pro-drug as an adjuvant. It further relates to the use of said AMP-kinase agonist or adenosine pro-drug in combination with a further adjuvant to diminish side effects of said adjuvant. It also further relates to the use of said AMP-kinase agonist or adenosine pro-drug as an anti-inflammatory compound.

In a third aspect, the present invention relates to a vaccine comprising an immune response eliciting compound and an AMP-kinase agonist or an adenosine pro-drug as an adjuvant and optionally a further adjuvant.

In a fourth aspect the present invention relates to a method for enhancing the immune response comprising co-administration of an immune response eliciting compound with an AMP-kinase agonist or an adenosine pro-drug. The present invention further relates to a method for decreasing the anti-inflammatory effect of a vaccine comprising co-administration of an immune response eliciting molecule and a therapeutically effective amount of an AMP-kinase agonist or an adenosine pro-drug.

### DETAILED DESCRIPTION OF THE INVENTION

The present invention relates to a composition comprising an immune response eliciting compound and an immune response enhancing compound chosen from the group of AMP-activated protein kinase (AMP-kinase) agonists and/or adenosine pro-drugs.

According to another embodiment, the present invention relates to a composition comprising an immune response eliciting compound and an immune response enhancing compound, said immune enhancing compound chosen from the group of AMP-activated protein kinase (AMP-kinase) agonists.

The term “immune response eliciting compound” or “immune response eliciting molecule” as used herein in the present invention is to be understood as any compound to which a receptor of the immune system, such as an antibody, can bind, and which can act as a target against which an immune response is induced. Preferably, said “immune response eliciting molecule” is an antigen.

According to a preferred embodiment the present invention relates to a composition as described above wherein said immune response eliciting molecule is of bacterial, viral or parasitical origin, more preferably a bacterial, viral, vegetable or parasitical antigen.

According to another embodiment, the present invention relates to a composition as described above wherein said immune response eliciting compound is an allergen.

According to another embodiment, the present invention relates to a composition as described above wherein said immune response eliciting compound is a live or attenuated microorganism.

The terms “antigen” or “antigenic material” which are used interchangeably herein include one or more non-viable immunogenic agents of bacterial, viral, plant, parasitical or other origin. The immune response eliciting component of the compositions of the invention may consist of a dried powder, an aqueous solution, an aqueous suspension and the like, including mixtures of the same, containing a non-viable immunogenic agent or agents. For instance the aqueous phase may be in the form of a vaccine in which the antigen is dissolved in a balanced salt solution, physiological saline solution, phosphate buffered saline solution, tissue culture fluids, or other media in which the organism may have been grown. The aqueous phase also may contain preservatives and/or substances such as those conventionally incorporated in vaccine preparations.

The antigen may be in the form of purified or partially purified antigen derived from bacteria, viruses, plants, parasites or their products, or extracts of bacteria, viruses, plants or parasites; or the antigen may be an allergen such as pollens, dusts, danders, or extracts of the same; or the antigen may be in the form of a poison or a venom derived from poisonous insects or reptiles. In all cases, the antigens will be in the form in which their toxic or virulent properties have been reduced or destroyed and which when introduced into a suitable host will either induce active immunity by the production therein of antibodies against the specific micro-organisms, extract, or products of micro-organisms used in the preparation of the antigen, or, in the case of allergens, they will aid in alleviating the symptoms of the allergy due to the specific allergen. The antigens can be used either singly or in combination, for example, multiple bacterial antigens, multiple viral antigens, multiple mycoplasmal antigens, multiple parasitical antigens, multiple bacterial or viral toxoids, multiple allergens or combinations of any of the foregoing products can be combined in the aqueous phase of the composition of this invention. Antigens of particular importance are derived from bacteria such as B. pertussis, Leptospira pomona, and icterohaemorrhagiae, S. paratyphi A and B, C. diphtheriae, C. tetani, C. botulinum, C. perfringens, C. feseri, and other gas gangrene bacteria, B. anthracis, P. pestis, P. multocida, V. cholerae, Neisseria meningitidis, N. gonorrheae, Hemophilus influenzae, Treponema pollidum, and the like; from viruses as polio virus (multiple types), adenovirus (multiple types), parainfluenza virus (multiple types), measles, mumps, respiratory syncytial virus, influenza (various types), shipping fever virus (SF4), Western and Eastern equine encephalomyelitis, Japanese B. encephalomyelitis, Russian Spring Summer encephalomyelitis, hog cholera virus, Newcastle disease virus, fowl pox, rabies, feline and canine distemper and the like viruses, from rickettsiae as epidemic and endemic typhus or other members of the spotted fever group, from various spider and snake venoms or any of the known allergens, for example, from ragweed, house dust, pollen extracts, grass pollens, and the like.

The “immune response eliciting compound” may also be a complete or part of a microorganism or organism, such as a live or attenuated microorganism or organism.

The expression “immune response enhancing” or “immuno-potentiating” can include administration of an agent effecting an increase in the rate at which the immune response develops, an increase in the intensity or level of the response, a prolongation of the response, or the development of a response to an otherwise non-immunogenic substance.

The agents that are known to enhance immune responses are generally termed adjuvants and can be grouped into two general classes: (1) those providing general potentiation; i.e., substances that enhance cellular and/or humoral immune responses to a wide variety of antigens, and (2) those providing specific potentiation, i.e., substances that enhance specific responses to certain antigens only. Substances that can act as class (1) adjuvants can be grouped into the following categories: (1) water and oil emulsions, e.g., Freund's adjuvant, (2) synthetic polynucleotides, (3) hormones, drugs and cyclic nucleotides, (4) endotoxins, (5) proteiriaceous lymphokines and monokines, e.g., interleukins.

Throughout the description the expressions “immune response enhancing agent”, “immunogenicity enhancing compound”, “immuno-stimulants” and “immuno-potentiating agents” will be used interchangeably and having the same meaning.

An immuno-potentiated state can be illustrated by the bodily condition after vaccination, wherein the immune response is already enhanced due to an antigenic response, but could be beneficially further enhanced to provide an improved degree and/or duration of immunity. Immuno-potentiation can occur in animals that exhibit a normal immune response as well as in animals that exhibit a compromised immune response. In the latter situation, immuno-potentiation is relative to the immuno-compromised status of the host animal, and rather than enhancing the response to supernormal levels, a protective degree of immunity (i.e., nearly normal levels) is sought and is referred to as immuno-reconstitution. References to immuno-enhancements hereinafter are to be understood to include immuno-reconstituted effecting an increase in the rate at which the immune response develops.

According to a preferred embodiment, the present invention relates to a composition wherein the AMP-kinase agonist is an AMP mimetic or adenosine pro-drug or derivative thereof, characterized in that said AMP mimetic or adenosine pro-drug or derivative thereof stimulates AMP-kinase

AMP-activated protein kinase (AMP-Kinase) belongs to a group of enzymes which, using ATP, phosphorylate proteins at serine or occasionally threonine residues. Best known in this group are cyclic AMP-dependent protein kinase (cAPK), Ca++/calmodulin-dependent protein kinases, and protein kinase C. Protein kinases are components of the transduction mechanisms whereby hormones and other factors regulate physiological functions. Their action elicits conformational changes that modify either the catalytic activity of enzymes or the function of other regulatory proteins. The conformational changes induced by phosphorylation can be reversed by protein phosphatases, of which several types have been characterized in recent years.

AMP-activated protein kinase (AMP-kinase) agonists are molecules that can mimic the activating affect of AMP on said AMP-kinase. These agonists are usually structural analogues of AMP such as AICA-riboside (5-amino 4-imidazole carboxamide riboside). AICA-riboside is a purine nucleoside analogue, which when metabolized by cells yields a compound, ZMP which activates AMP-kinase. AICA-riboside has been shown to activate AMP-kinase in skeletal muscle and liver and by doing so it exerts a wide variety of metabolic effects on these tissues. In muscle, it reproduces many of the effects of exercise, including phosphorylation and inhibition of acetyl CoA carboxylase, and increases in fatty acid oxidation and glucose transport. Once activated, the AMP-kinase initiates energy-conserving measures (such inhibition of most macromolecules biosynthesis) and mobilizes the catabolism of alternative carbon sources such as lipids. AMP-kinase appears therefore to protect cells against metabolic stress.

Said AMP mimetics or adenosine pro-drugs or derivatives thereof for use in the present invention include, for example, AICA-riboside (5′-amino-imidazolecarboxamide riboside), SAICAR (5-amino-4-imidazole-N-succinocarboxamide riboside), ZMP (5-aminoimidazole-4-carboxamide-1-beta-D-ribofuranosyl-5′-monophosphate), 6-MPR (6-mercaptopurine riboside), AMP analogues or a derivative of any of the above mentioned compounds. According to further embodiment, also analogs of any of the above mentioned compounds are meant, and any pro-drugs which can be used to produce such AICA-riboside, such as SAICAR, ZMP, and analogs thereof, within an individual body and pro-drugs thereof, which can produce AMP mimetics or adenosine within a body of the individual being treated. Other examples include di- and triphosphate derivatives of the compounds cited herein. By “base” is meant a compound which when phosphorylated is a nucleotide and serves as an AMP mimetic. The term “pro-drug” refers to compounds which are derivatives of a parent compound (such as AICA-riboside) which have been derivatized to assist the parent compound in getting to the desired locus of action. The derivatized portion of the pro-drug is cleaved or activated to give the parent compound either in transit or at the desired locus. Typically a pro-drug may allow the parent compound to cross or better cross a biological barrier such as the gut epithelium, the cellular plasma membranes or the blood brain barrier, at which point it is cleaved to give the parent compound.

According to a more preferred embodiment, said immune response enhancing compound is selected from the group consisting of AICA-riboside, SAICAR, ZMP, 6-MPR (6-mercaptopurine riboside), AMP analogues and adenosine pro-drugs, analogues and derivatives of any of said compounds. Preferably, said immune response enhancing compound is characterized in that it stimulates or activates AMP-kinase.

Several methods to evaluate the ability of a given compound or procedure to activate the AMPK have been described. These include:

1. Evaluation of the ability of AMPK, a serine/threonine protein kinase, to phosphorylate in vivo or in vitro a given substrate. Typically, in these experiments the substrate is a protein or a peptide containing a sequence similar to the sequence surrounding the site phosphorylated by AMPK on its natural substrates. The AMARA peptide (AMARAASAAALARRR) and the SAMS peptide (HMRSAMSGLHLVKRR) are typically used as substrates in these studies. Briefly, AMPK-containing cell extracts or purified forms of the AMPK are incubated in the presence of a substrate, an adequate reaction mix and a tracer, usually a phosphorylated form of ATP (See Davies et al., S. P., Carling, D. and Hardie, D. G. (1989) Eur. J. Biochem. 186, 123-128 for an example).

2. AMPK has been found to be a heterotrimeric complex composed of a catalytic subunit (alpha) and two regulatory subunits (beta and gamma). Activation of AMPK leads to phosphorylation of the catalytic unit (the alpha unit) on a given residue (Thr 172) that can be identified using specific antibodies reacting with the phosphorylated form of the AMPK alpha unit. Briefly, the ability of a given compound or procedure to activate the AMPK can be evaluated by its ability to induce phosphorylation of the Thr 172 residues on the AMPK catalytic subunit (See Fryer L G, Parbu-Patel and Carling, J. Biol. Chem 2002 277:25226 as a recent example).

3. Further, AMPK is known to regulate glucose uptake in several cell lines and tissues. Upon activation, AMPK induces glucose uptake that can be monitored using a radioactive ligand (glucose or glucose mimetic). Briefly, the ability of a given compound or procedure to activate the AMPK can be evaluated by its ability to increase glucose uptake in a given cell line or tissue (See Abbud W, Habinowski S, Zhang J Z, Kendrew J, Elkairi F S, Kemp B E, Witters L A, Ismail-Beigi F, Arch Biochem Biophys 2000 380:347).

According to a yet more preferred embodiment said immune response enhancing compound is AICA-riboside. According to another embodiment said immune response enhancing compound is 6 MPR (6-mercaptopurine riboside).

AICA-riboside acts as a prototype adenosine-regulating agent or a cell-permeable activator of AMP-activated protein kinase. When added in culture or injected in vivo, AICA-riboside can be taken up by cells and phosphorylated intracellularly into monophosphate form termed ZMP. Exposure of cells to AICA-Riboside causes therefore an intracellular accumulation of ZMP. ZMP is structurally related to AMP, and has been shown to mimic the effects of AMP on the AMP-kinase (not to be confused with the better-known cAMP-dependent protein kinase or PKA). Consequently, addition of AICA-riboside to cells causes the activation of the AMP-kinase enzyme. The AMP-kinase enzyme acts as a metabolic sensor that monitors intracellular AMP levels. High AMP levels, indicative of a metabolic stress, activate this enzyme. Indeed, under optimal conditions, AMP intracellular levels are very low and the ATP/AMP ratio is in the order of 100. Under metabolic stress (such as lack of adequate nutrient supply, hypoxia or inhibition of mitochondria activity), ATP is converted to ADP and subsequently to AMP, causing an accumulation of intracellular AMP. Once activated by high levels of intracellular AMP, the AMP-kinase initiates energy-conserving measures (such inhibition of most macromolecules biosynthesis) and mobilizes the catabolism of alternative carbon sources such as lipids. AMP-kinase appears therefore to protect cells against metabolic stress. Moreover, addition of AICA-riboside to cells in vivo and in vitro causes the accumulation of extracellular adenosine, a nucleotide known to interact with several membrane receptors in most cell types (adenosine receptors). AICA-riboside can be metabolized into adenosine, which then accumulates in the extracellular milieu.

Although the precise mechanism(s) by which AICA-riboside augments antibody responses in vivo is presently unknown, some points are noteworthy in light of the present invention. A single co-injection of AICA-riboside and antigen is sufficient to induce a prolonged elevation of specific antibodies production during the primary and secondary response, whereas an injection of AICA-ribose alone does not induce an immune response. A qualitative change of the humoral response is also observed, as immunoglobulins of the IgG isotype (generally detected only during the secondary response) are already detected during the primary response in animals treated with antigen/AICA-riboside formulations. Studies performed in mice indicate that when mixed in aqueous solutions with protein antigens (several antigens have been tested) this compound boosted serum antibody response in mice five to ten folds above unadjuvanted antigens, therefore showing an unexpected synergetic effect. AICA-riboside has been proved effective when delivered together with antigens via different administration routes including intra-peritoneally, intra-veinously, sub-cutaneously or intra-muscularly. The immuno-potentiating effect of AICA-riboside in combination with said antigen is as good when the compounds are administered mixed together (co-administered) as when administered separately or sequentially. AICA-riboside does not induce local inflammation upon administration to an individual. The use of this agent therefore probably precludes local pain after injection. Furthermore, studies have demonstrated that the drug was well tolerated in humans at all dose levels (10 to 100 mg/kg), with no apparent side effects. Moreover, no enhancing effect of AICA-riboside on the in vitro response of murine lymphocytes was observed. In particular, AICA-riboside did not affect the secretion of cytokines in response to all agents tested, which included bacterial mitogens, lectins or protein antigens. These data suggest that AICA-riboside does not act in vivo by activating immune cells to cytokine secretion. Accordingly, no local inflammatory response was observed following injection of AICA-riboside into mice.

In any event, the proposal is made that activation of the AMP-kinase or the accumulation of extracellular adenosine represents an effective means to enhance antibody responses in both human and animal models. AICA-riboside does not activate immune cells to cytokine secretion and can be administered as an aqueous solution, suggesting that it may induce only minimal inflammation and/or pain. Moreover, due to its small molecular weight, this molecule is not expected to be immunogenic. Consequently said AICA-riboside may be used as an anti-inflammatory compound.

Further, the present invention also relates to immune enhancing compounds which indirectly stimulate AMP-kinase. AMPK can be activated by conditions leading to a rise in cellular AMP or rise in cellular AMP/ATP ratio. While a rise in AMP can be mimicked by AMP analogues such as AICAR, a rise in the AMP/ATP ratio can be induced in the cell by interfering with ATP synthesis. Inhibition of ATP synthesis can be achieved, for instance, using drugs that affect generation of ATP such as oligomycin, an inhibitor of mitochondrial oxidative phosphorylation or 2-deoxyglucose, an inhibitor of the glycolytic pathway (see Krause et al., 2002, Eur J. Biochem., 269(15):3751-9 and Horman et al., 2002, of Protein Curr Biol. 12 (16):1419). This embodiment of the invention is illustrated in Example 11 describing an experiment wherein the effect of a compound is shown to activate AMPK in vivo by a mechanism distinct from the generation of AMP agonists. The experiment depicted in FIG. 11 demonstrates that injection of an ATP-synthesis inhibitor (oligomycin) increases antibody responses, providing an additional example in favour of the idea that increased AMPK leads to enhanced antibody responses.

The immune response enhancing compound useful according to the present invention may have asymmetric centers, occur as racemates, racemic mixtures, and as individual diastereoisomers, with all possible stereochemical isomers including optical isomers, being included in the present invention.

The present invention when referring to immune response enhancing compounds such as the AMP mimetics or pro-drugs or derivatives thereof or the adenosine pro-drugs, as cited herein, also includes within its scope not just the specific compound(s) listed or described, but also alternative forms of the compound such as pharmaceutically acceptable salts, solvates, hydrates, and the like. The pharmaceutically acceptable salts of the compounds of this invention include the conventional non-toxic salts of the compounds as formed, e.g., from non-toxic inorganic or organic acids. For example, such conventional non-toxic salts include those derived from inorganic acids such as hydrochloric, hydrobromic, sulfuric, sulfamic, phosphoric, nitric and the like: and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, isethionic, trifluoroacetic and the like.

The composition of the present invention is preferably a pharmaceutical composition and may further include pharmaceutically acceptable carrier, thickeners, diluents, buffers, preservatives, surface active agents, liposomes, or lipid formulations, and the like. Pharmaceutically acceptable carriers may include sterile aqueous or non-aqueous solutions, suspensions, and emulsions suitable for ingestion, inhalation, or administration as a suppository to the rectum or vagina. Examples of non-aqueous solvents are propylene glycol, polyethylene glycol, vegetable oils such as olive oil, and certain organic esters such as ethyl oleate. Aqueous carriers include water, alcoholic/aqueous solutions, emulsions or suspensions, including saline and buffered media. Said pharmaceutical composition may also include one or more additional active ingredients such as chemotherapy agents, antimicrobial agents, anti-inflammatory agents, anesthetics, and the like.

Said pharmaceutical composition may be administered to an individual in a number of ways depending on whether local or systemic treatment is desired, and on the area to be treated.

The term “individual” as used herein refers to an animal such as vertebrates. Examples of these vertebrates include fish, birds, and mammal. The individual will preferably be a human, but may also be a domestic livestock, poultry, fish, laboratory or pet animals. Administration may be topically including on the skin, ophthalmically, vaginally, rectally, intranasally, orally, by inhalation, or parenterally, for example by intravenous drip, subcutaneous, intratumor, intraperitoneal, intralymphatic or intramuscular injection. The preferred mode of administration is parenterally.

According to another embodiment, the present invention relates to the use of an AMP-kinase agonist or an adenosine pro-drug as an immune response enhancing compound. According to yet another embodiment, the present invention relates to the use of an AMP-kinase agonist or an adenosine pro-drug as an adjuvant, preferably an adjuvant in a vaccine or vaccine composition.

The present inventors surprisingly found that the use of AMP-kinase agonists or adenosine pro-drugs and more specific, AICA-riboside diminished the local inflammatory response at the place of injection. Therefore, said molecules can also be used as anti-inflammatory compounds whenever a pharmaceutical or therapeutical solution is administered to an individual. The present invention thus relates to the use of AMP-kinase agonists or adenosine pro-drugs, preferably AICA-riboside or 6 MPR (6-mercaptopurine riboside) as anti-inflammatory compound. A more preferred use of said AMP-kinase agonist or said adenosine pro-drug as an adjuvant or as anti-inflammatory compound is in combination with a further adjuvant, for instance a known adjuvant (such as but not restricted to alum) to diminish side effects of said further adjuvant.

Adverse event associated with vaccination in both human and animal models often include erythrema and swelling at the injection site, and fever (see Ada G, N Engi J Med 2001, 345:1042). Freund's adjuvant, a prototypic emulsion adjuvant has for example been restricted to use in experimental animals due to its high reactogenicity. Even aluminium hydroxide, an adjuvant widely used in human and veterinary vaccines may not be considered as devoid of side effects, as suggested by recent reports describing the possible induction of fibrosarcomas in cats by this aluminium salt (Lester S et al, J Am Anim Hosp Assoc 1996, 32:91 and Burton G and Mason K V, Aust Vet J 1997, 75:100). Muramyl dipeptide (MDP) is a synthetic compound with strong adjuvant activity. Numerous studies have illustrated strong adverse reactions associated with MDP-containing vaccines (see Allison A C and Byers N E Mol Immunol 1991 28:279, Keitel W et al, Vaccine 1993, 11:909 and Hoffman S L et al, Am J Trop Med Hyg 1994, 51:603). In addition to the well know early reactions, additional side effects associated with mineral oil and salt adjuvants might derive from their poor ability to be metabolized in vivo, raising concern about possible long-term effects of their residues.

The AMP-kinase agonists or adenosine pro-drugs according to the invention have proven not to exert, or to exert at a lesser extent, the side effects encountered with other known and generally accepted adjuvants, as demonstrated for instance in Examples 8 and 9.

According to a preferred embodiment the present invention relates to any of the above described uses wherein said AMP-kinase agonist or adenosine pro-drug is selected from the group consisting of AICA-riboside, AICA base, SAICAR, ZMP, 6-MPR (6-mercaptopurine riboside), AMP analogues or AMP mimetics and pro-drugs, analogues and derivatives of any of the aforementioned compounds. Preferably, said AMP analogue or AMP mimetic and said adenosine pro-drug stimulate or activate AMP-kinase.

More preferably, said AMP-kinase agonist or adenosine pro-drug for the uses as described above is AICA-ribose or 6-MPR (6-mercaptopurine riboside).

In another aspect of the present invention said AMP-kinase agonists or adenosine pro-drugs may be used as a novel type of adjuvant for the preparation of vaccines.

In an even more specific embodiment, the present invention relates to the use of AICA-riboside as an adjuvant in or for a vaccine. In a further specific embodiment, the present invention relates to the use of 6-MPR (6-mercaptopurine riboside)-as an adjuvant in or for a vaccine.

Therefore, the present invention also relates to a vaccine comprising an immune response eliciting molecule and an AMP-kinase agonist or an adenosine pro-drug as an adjuvant, optionally comprising a further (or second) adjuvant. According to a preferred embodiment, said AMP-kinase agonist or adenosine pro-drug used in the vaccine as an adjuvant, is an AMP mimetic or pro-drug or derivative thereof. In a more preferred embodiment, said AMP-kinase agonist or adenosine pro-drug is selected from the group consisting of AICA-riboside (5-aminoimidazole-4-carboxamide riboside), SAICAR (5-amino-4-imidazole-N-succinocarboxamide riboside), ZMP (5-aminoimidazole-4-carboxamide-1-beta-D-ribofuranosyl-5′-monophosphate), 6-MPR (6-mercaptopurine riboside), AMP analogues and derivatives of any of said compounds. Yet more preferably, the invention relates to a vaccine comprising AICA-Riboside or 6 MPR (6-mercaptopurine riboside) as an adjuvant.

The vaccine or the composition according to the invention may suitably be provided in the form of a spray, an aerosol, a mixture, tablets (entero-or not-enterocoated), capsule (hard or soft, entero-or not-enterocoated), a suspension, a dispersion, granules, a powder, a solution, an emulsion, chewable tablets, tablets for dissolution, drops, a gel, a paste, a syrup, a cream, a lozenge (powder, granulate, tablets), an instillation fluid, a gas, a vapor, an ointment, a stick, implants (ear, eye, skin, nose, rectal, or vaginal), sterile injectable preparations, for example, sterile injectable aqueous or oleaginous suspensions, vagitories, suppositories, or uteritories suitable for administration via the parenteral (intravenous, subcutaneous, intratumor, intraperitoneal, intralymphatic or intramuscular injection.), oral, nasal, vaginal, sublingual, ocular, rectal, urinal, intramammal, pulmonal, otolar, or buccal route.

The invention also relates to a method for enhancing the immune response, and particularly an antigen-specific immune response, comprising co-administration of an immune response eliciting molecule with an AMP-kinase agonist or an adenosine pro-drug. The method is based upon the finding that AICA-riboside, and related analogs (which are structural mimetics of AMP), are effective in enhancing the immune response elicited by a molecule. These compounds have their effect by stimulating AMP-kinase (AMP-kinase agonists). Other compounds have their effect by being metabolized into adenosine (adenosine pro-drugs). In another aspect the present invention further relates to a method for decreasing the anti-inflammatory effect of a vaccine comprising co-administration of an immune response eliciting molecule and a therapeutically effective amount of an AMP-kinase agonist or an adenosine pro-drug.

The “therapeutically effective amount” of said above-described AMP-kinase agonist and/or adenosine pro-drug relates to the amount or quantity of compound according to the invention which is sufficient to elicit the required or desired therapeutic response, or in other words, the amount which is sufficient to elicit an appreciable biological response when administered to an individual (patient).

The above-described method includes introducing into an individual, separately or together (co-administered), an immune response eliciting molecule and a therapeutically effective amount of an AMP-kinase agonist and/or an adenosine pro-drug. Said AMP-agonist is preferably an AMP mimetic, or pro-drug which is a compound which can be administered to generate an AMP mimetic in vivo or derivative thereof which stimulates AMP-kinase. Said adenosine pro-drug includes compounds, which are precursor or metabolized into adenosine. It includes compounds which upon administration are activated to produce said AMP mimetic or to produce adenosine, for example esters which can be cleaved, or nucleosides which can be phosphorylated or bases which can be phosphoribosylated to form said AMP mimetic or adenosine. According to a more preferred embodiment said AMP-kinase agonist and/or adenosine pro-drug is selected from the group consisting of AICA-riboside, AICA base, SAICAR, ZMP, 6-MPR (6-mercaptopurine riboside), AMP analogues, and pro-drugs, analogues and derivatives thereof, yet most preferably AICA-riboside or 6-MPR.

The present invention further relates to a product containing an immune response eliciting molecule and an immune response enhancing compound chosen from the group of AMP-activated protein kinase (AMP-kinase) agonists and/or adenosine pro-drug, as a combined preparation for mixed or separate or sequential use in immuno-enhandng therapies. According to a preferred embodiment said AMP-kinase agonist or adenosine pro-drug is selected from the group consisting of AICA-riboside, AICA base, SAICAR, ZMP, 6-MPR (6-mercaptopurine riboside), AMP analogues and derivatives of any of said compounds. Preferably, said adenosine pro-drugs or derivatives thereof stimulate AMP-kinase.

In a more preferred embodiment said AMP-kinase agonist or adenosine pro-drug is AICA-riboside or 6-MPR (6-mercaptopurine riboside).

The invention will be more readily understood by reference to the following examples and figures, which are included merely for purposes of illustration of certain aspects and embodiments of the present invention and are not intended to limit the invention.

### BRIEF DESCRIPTION OF THE FIGURES

FIG. 1 illustrates the immuno-stimulatory properties of AICA-riboside when co-administered with an antigen: the hapten-protein conjugate nitrophenylacetyl-keyhole limpet hemocyanin (NP-KLH).

FIG. 2 illustrates the effect of AICA-riboside as an adjuvant on the primary and secondary antibody responses, when co-administered with the hapten conjugate p-azophenylarsonat-keyhole limpet hemocyanin (Ars-KLH).

FIG. 3 illustrates the long-lasting effect of AICA-riboside as an adjuvant.

FIG. 4 illustrates the effect of AICA-riboside as an adjuvant on the primary and secondary antibody response to the protein antigen human gamma globulin (HGG).

FIG. 5 illustrates the effect of AICA-riboside as an adjuvant over a wide range of doses.

FIG. 6 illustrates that AICA-riboside acts as an adjuvant when injected simultaneously with an antigen.

FIG. 7 illustrates that the effect of AICA-riboside as an adjuvant requires the presence of CD4 positive T cells.

FIG. 8 illustrates that AICA-riboside acts as an adjuvant without inducing the swelling of the site of injection-draining lymph nodes.

FIG. 9 illustrates that AICA-riboside injection does not cause a local inflammatory response.

FIG. 10 illustrates the effect of AICA-riboside and 6-mercaptopurine riboside as adjuvants on the primary antibody response to Ars-KLH.

FIG. 11 illustrates the effect of AICA-riboside and oligomycin (an inhibitor of mitochondrial ATP synthase) on the primary and secondary antibody response to a hapten-protein antigen (Ars-KLH).

### EXAMPLES

### Example 1

Immuno-stimulatory properties of AICA-riboside when co-administered with an antigen: the hapten-protein conjugate nitrophenylacetyl-keyhole limpet hemocyanin (NP-KLH).

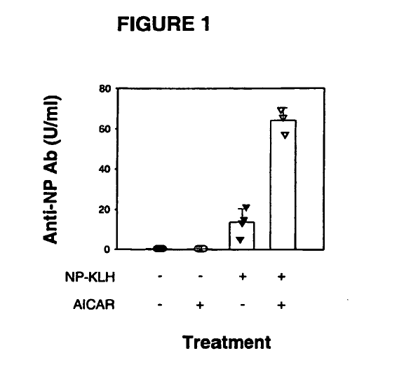

BALB/c mice were injected intraperitoneally with 200 μl of saline, aqueous solution (phosphate buffer solution), or with 200 μl of phosphate buffer solution containing AICA-riboside (10 mg), NP-KLH (100 μg) or NP-KLH (100 μg)+AICA-riboside (10 mg). Height days after immunization, mice were bled and serum levels of antigen (NP)-specific antibodies were determined by ELISA according to standard procedure using isotype-specific reagents. As illustrated FIG. 1, this experiment demonstrates that co-administration of AICA-riboside with an antigen causes an increased antigen-specific antibody response when compared to antigen-administration alone. Moreover, the experiment demonstrates that administration of AICA-riboside alone does not lead to an increased, antigen-specific antibody response.

### Example 2

Effect of AICA-riboside as an adjuvant on the primary and secondary antibody: responses, when co-administered with the hapten conjugate p-azophenylarsonat-keyhole limpet hemocyanin (Ars-KLH).

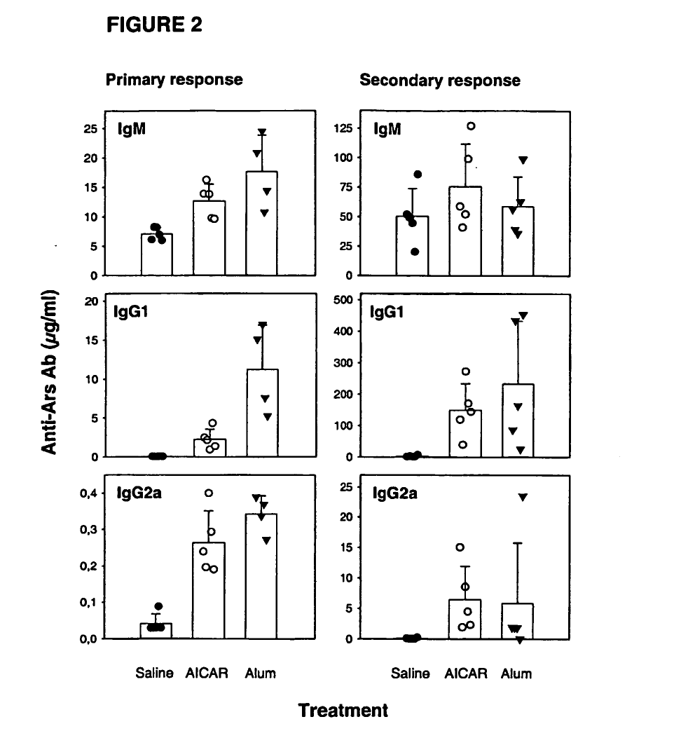

Three groups of BALB/c mice received an intraperitoneal injection of Ars-KLH (100 μg), Ars-KLH (100 μg)+AICA-riboside (10 mg) or Ars-KLH (100 μg)+Alum (50 μl). 21 days after immunization, mice were bled and serum levels of antigen (Ars)-specific antibodies were determined by ELISA as previously described (primary response). On day 22, all mice received a second injection (boost) of Ars-KLH (100 μg) i.p. Mice were bled 8 days after the antigen boost and serum levels of Ars-specific antibodies were determined by ELISA (secondary response). The results are illustrated in FIG. 2.

This experiment demonstrates that co-administration of AICA-riboside with an antigen leads to increased levels of antigen-specific IgG antibodies when compared to control mice immunized with antigen in the absence of adjuvant, leaving the antigen-specific IgM response unaffected. The experiment also demonstrates that AICA-riboside displays immuno-stimulatory properties comparable to the Alum adjuvant.

### Example 3

Long-lasting effect of AICA-riboside as an adjuvant.

Mice immunized according to the protocol described in example 2 were bled 120 days after the first encounter with the antigen (in the presence or absence of adjuvant) and serum levels of antigen specific antibodies were determined by ELISA as described previously (secondary response, day 120).

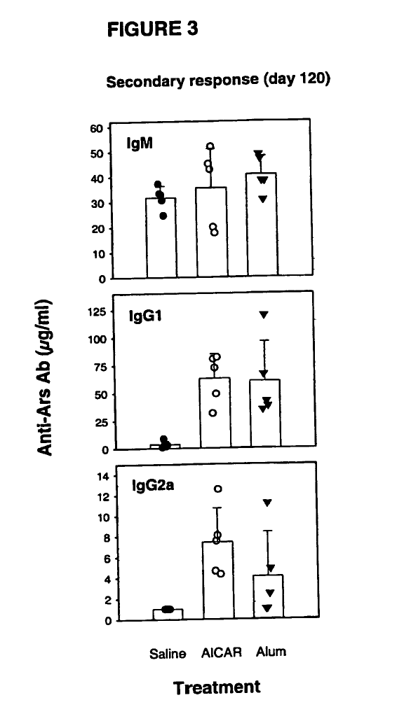

This experiment demonstrates that co-administration of AICA-riboside with an antigen leads to a long-lasting increase in antigen-specific response when compared to animals injected with antigen alone (FIG. 3).

### Example 4

Effect of AICA-riboside as an adjuvant on the primary and secondary antibody response to the protein antigen human gamma globulins (HGG).

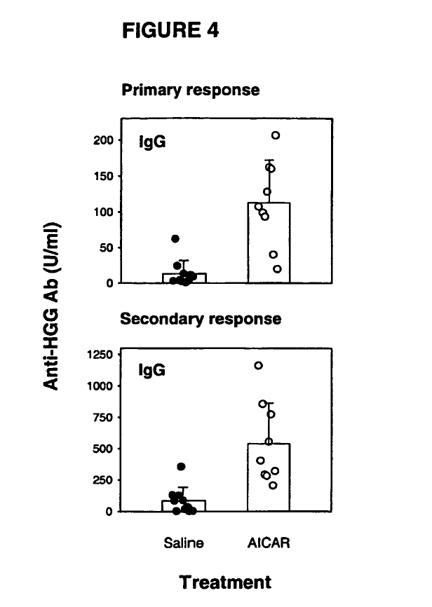

BALB/c mice received an intraperitoneal injection of HGG (15 μg) or HGG (15 μg)+AICA-riboside (10 mg). 16 days after immunization, mice were bled and serum levels of HGG-specific antibodies were determined by ELISA as described. On day 25, the two groups of mice received respectively a boost of HGG (15 μg) and of HGG (15 μg)+AICA-riboside (10 mg) intraperitoneally. Mice were bled 10 days after the boost and serum levels of HGG-specific antibodies were determined by ELISA as described previously (secondary response). The results are illustrated in FIG. 4.

This experiment demonstrates that AICA-riboside augments the antibody response to a protein antigen of non-infectious origin.

### Example 5

AICA-riboside acts as an adjuvant over a wide range of doses.

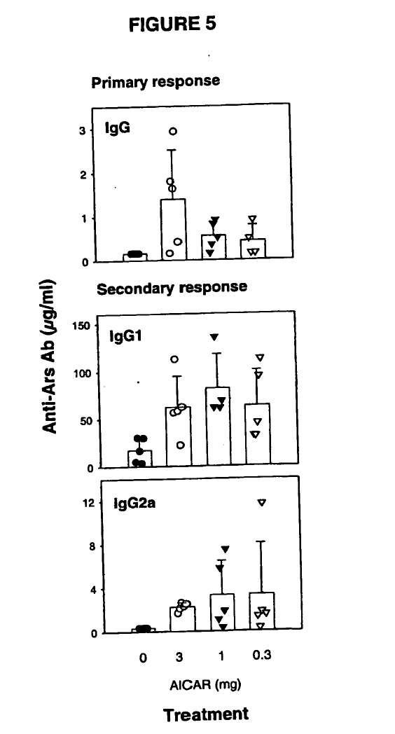

BALB/c mice received an intraperitoneal injection of Ars-KLH (100 μg) or Ars-KLH (100 μg)+AICA-riboside at concentrations ranging from 3 to 0.3 mg. 21 days after immunization, mice were bled and serum levels of antigen (Ars)-specific antibodies were determined by ELISA according to standard procedure using isotype-specific reagents (primary response). On day 22, all mice received a boost of Ars-KLH (50 μg) intraperitoneally. Mice were bled 14 days after the antigen boost and serum levels of Ars-specific antibodies were determined by ELISA as described previously (secondary response). The results are illustrated in FIG. 5.

This experiment demonstrates that AICA-riboside can act as an adjuvant over a wide range of doses (including the dose of 100 mg/kg that have been used safely in humans). Note that the optimal immuno-stimulatory dose of AICA-riboside may depend on several factors including antigen nature, antigen dose, host species.

### Example 6

AICA-riboside acts as an adjuvant when injected simultaneously with an antigen.

BALB/c mice received an intraperitoneal injection of AICA-riboside (10 mg) one day before (d-1), the same day (d0) or the day after (d+1) the intraperitoneal injection (in a separate site) of Ars-KLH (100 μg). A fourth group (n.t.) received an intraperitoneal injection of phosphate buffer solution the day of the antigen administration. 20 days after immunization, mice were bled and serum levels of Ars-specific antibodies were determined by ELISA according to standard procedures using isotype-specific reagents (primary response). On day 21, all mice received a boost of Ars-KLH (50 μg) intraperitoneally. Mice were bled 14 days after the antigen boost and serum levels of Ars-specific antibodies were determined by ELISA as described previously (secondary response).

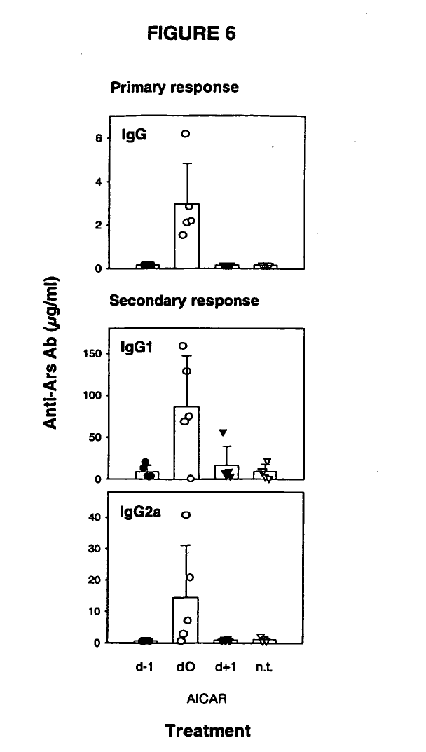

This experiment demonstrates that the adjuvant effect of AICA-riboside is only manifest when AICA-riboside is injected at the same moment with the antigen (FIG. 6). Moreover, the experiment demonstrates that AICA-riboside and antigen do not require in vitro mixing prior to in vivo administration and can be administered separately.

### Example 7

Effect of AICA-riboside as an adjuvant requires the presence of CD4 positive T cells.

BALB/c mice, were treated or not with anti-CD4 antibodies (two intraperitoneal injections of 200 μl of the depleting rat anti-mouse anti-CD4 antibody, clone GK1.5, on days 0 and 5). On day 2 mice were injected intraperitoneally with NP-KLH (100 μg) or NP-KLH (100 μg)+AICA-riboside (10 mg). 10 days after immunization, mice were bled and serum levels of NP-specific antibodies were determined by ELISA according to standard procedures using isotype specific reagents.

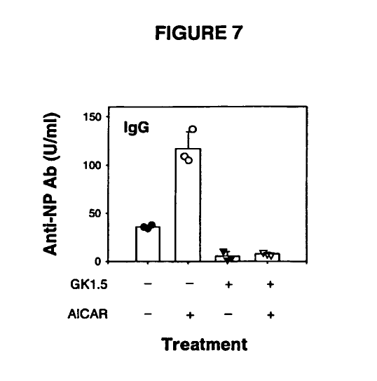

As illustrated FIG. 7, this experiment demonstrates that AICA-riboside does not augment the antigen-specific antibody responses in the absence of CD4-expressing T cells.

### Example 8

AICA-riboside acts as an adjuvant without inducing the swelling of the site of injection-draining lymph nodes.

BALB/c mice were injected with NP-KLH (25 μg), NP-KLH (25 μg)+AICA-riboside (2.5 mg) or NP-KLH (25 μg)+Alum (12.5 μg) in the two rear footpads. 8 days after injection, mice were sacrificed and their posterior lymph nodes were collected and immediately analyzed for size.

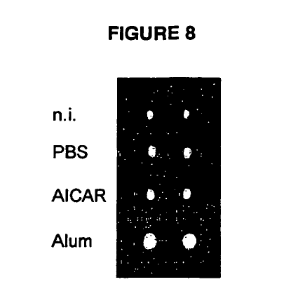

This experiment indicates that administration of AICA-riboside does not induce the swelling of the lymph node draining the injection site, as observed in response to adjuvants known to induce a local inflammatory reaction (such as Alum) (FIG. 8).

### Example 9

AICA-riboside injection does not cause a local inflammatory response.

BALB/c mice were injected with saline (50 μl PBS), AICA-boside (2.5 mg) or alum (12.5 μg) in the two rear footpads. 8 or 24 hours after treatment mice were sacrificed, and thin sections of the tissues surrounding the injection site (marked by a circle) were analyzed by immunohistochemistry. Inflammatory cells (mostly granulocytes and monocytes) were identified using an antibody directed to the murine GR-1 molecule.

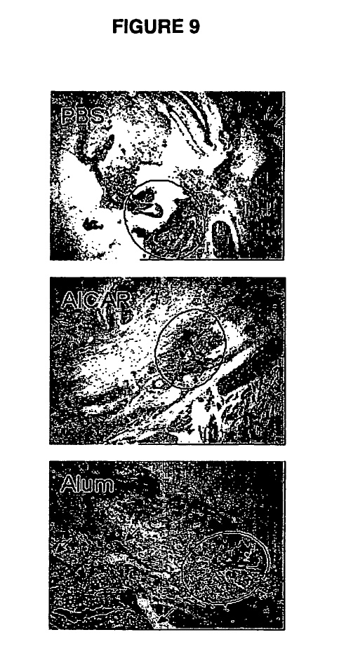

As illustrated FIG. 9, this experiment demonstrates that although mononuclear cells infiltrate the injection site in all sections, animals treated with saline or AICA-riboside display minimal GR-1-expressing cells recruitment to sites distant from the injection site, and display no tissue necrosis or abnormality. In marked contrast, mice treated with Alum display abundant cellular infiltrates (see arrows) and tissue necrosis.

### Example 10

Effect of AICA-riboside and 6-mercaptopurine (6-MPR) riboside as adjuvants on the primary antibody response to Ars-KLH.

BALB/c micereceived an intraperitoneal injection of Ars-KLH (100 μg), Ars-KLH+AICAR (3 mg) or Ars-KLH+6-MPR (3 mg). 19 days after Immunization, mice were bled and serum levels of antigen specific antibodies were determined by ELISA according to standard procedure using isotype-specific rat Mab.

Ars specific antibodies concentrations were respectively (μg/ml): Ars-KLH+saline: 0.04; Ars-KLH+AICAR: 1.607±0.521; Ars-KLH+6-MPR: 0.509 t 0.473.

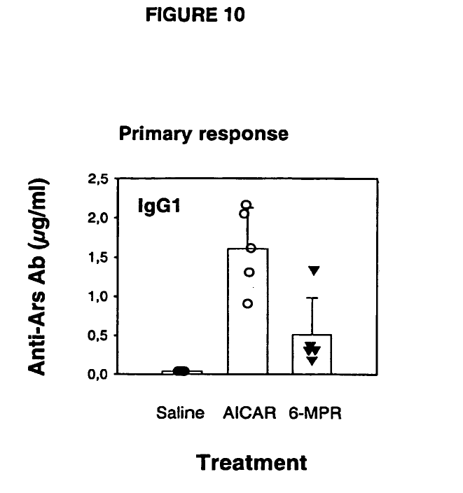

This experiment demonstrates that co-administration of AICA-riboside with an antigen and co-administration of 6-MPR with an antigen leads to increased levels of antigen-specific IgG antibodies when compared to control mice immunized with antigen in the absence of adjuvant (FIG. 10).

The above-describes experiments demonstrate clearly the synergetic action of an AMP-kinase agonist or an adenosine pro-drug with an antigen in enhancing an immune response when compared with the antigen alone. Moreover, the same synergetic enhancing effect is observed when the antigen and the AICA-riboside are administered mixed together (co-administered) or when they are injected separately. Furthermore, injection of AICA-riboside did not induce any inflammatory reaction, whereas injection of known adjuvants such as alum does provoke an inflammatory reaction at the site of injection.

### Example 11

Effect of AICA-riboside and oligomycin (an inhibitor of mitochondrial ATP synthase) on the primary and secondary antibody response to a hapten-protein antigen (Ars-KLH).

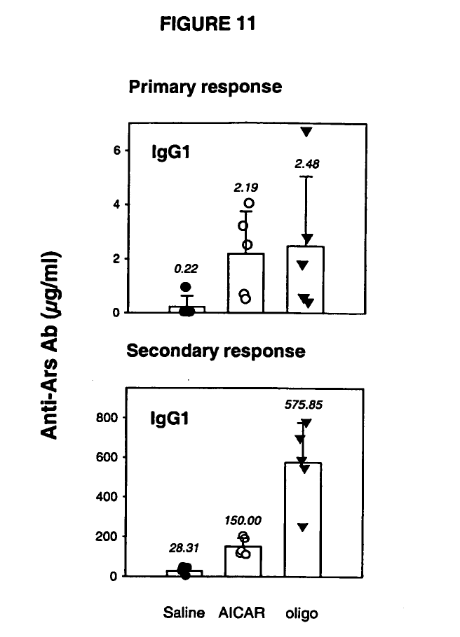

BALB/C mice were injected intraperitoneally with Ars-KLH (50 μg) mixed with saline, AICA-riboside (3 mg) or oligomycin (10 μg). 21 days after immunization, mice were bled and serum levels of Ars-specific antibodies (primary response) were determined by ELISA according to standard procedure using isotype-specific rat mAb. The level of IgG antibodies of the primary response is shown in the top panel of FIG. 11. On day 22, all mice received a second injection of Ars-KLH (50 μg) intrapertioneally. Mice were bled 14 days after the antigen boost and serum levels of Ars-KLH-specific antibodies (secondary response) were determined by ELISA. The level of IgG antibodies during the secondary response is shown in the bottom panel of FIG. 11.

This experiment demonstrates that co-administration of oligomycin with an antigen leads to increased levels of antigen-specific IgG antibodies when compared to control mice immunized with the antigen alone.

1. Use of an AMP-kinase agonist as an immune response enhancing compound.

2. Use of an AMP-kinase agonist as an adjuvant.

3. Use of an AMP-kinase agonist as an anti-inflammatory compound.

4. Use of an AMP-kinase agonist in combination with a further adjuvant to diminish side effects of said adjuvant.

5. Use according to any of claims 1 to 4 wherein said AMP-kinase agonist is an AMP mimetic or adenosine pro-drug or derivative thereof which stimulates AMP-kinase.

6. Use according to any of claims 1 to 5, wherein said AMP-kinase agonist is selected from the group consisting of AICA-riboside (5-aminoimidazole-4-carboxamide riboside), SAICAR (5-amino-4-imidazole-N-succinocarboxamide riboside), ZMP (5-aminoimidazole-4-carboxamide-1-beta-D-ribofuranosyl-5′-monophosphate), 6-MPR (6-mercaptopurine riboside), AMP analogues, and derivatives of any of said compounds.

7. Use according to any of claims 1 to 6, wherein said AMP-kinase agonist is AICA-riboside.

8. Use according to any of claims 1 to 6, wherein said AMP-kinase agonist is 6-MPR (6-mercaptopurine riboside).

9. A vaccine comprising an immune response eliciting molecule and an AMP-kinase agonist as an adjuvant, optionally further comprising a further adjuvant.

10. A vaccine according to claim 9, wherein said AMP-kinase agonist is selected from the group consisting of AICA-riboside (5-aminoimidazole-4-carboxamide riboside), SAICAR (5-amino-4-imidazole-N-succinocarboxamide riboside), ZMP (5-aminoimidazole-4-carboxamide-1-beta-D-ribofuranosyl-5′-monophosphate), 6-MPR (6-mercaptopurine riboside), AMP analogues, and derivatives of any of said compounds.

11. A vaccine according to claim 10, wherein said AMP-kinase agonist is AICA-riboside.

12. A vaccine according of claim 10, wherein said AMP-kinase agonist is 6-MPR (6-mercaptopurine riboside).

13. A method for enhancing the immune response comprising co-administration of an immune response eliciting molecule with an AMP-kinase agonist.

14. A method for decreasing the anti-inflammatory effect of a vaccine comprising Co-administration of a immune response eliciting molecule and a therapeutically effective amount of an AMP-kinase agonist.

15. A method according to any of claims 13 or 14 or combined 13 and 14, wherein said AMP-kinase agonist is selected from the group consisting of AICA-riboside (5-aminoimidazole-4-carboxamide riboside), AICA base (5-aminoimidazole-4-carboxamide), SAICAR (5-amino-4-imidazole-N-succinocarboxamide riboside), ZMP (5-aminoimidazole-4-carboxamide-1-beta-D-ribofuranosyl-5′-monophosphate), 6-MPR (6-mercaptopurine riboside), AMP analogues, and derivatives of any of said compounds.

16. A method according to claim 15, wherein said AMP-kinase agonist is AICA-riboside.

17. A method according to claim 15, wherein said AMP-kinase agonist is 6-MPR (6-mercaptopurine riboside).

18. A composition comprising an immune response eliciting compound and an immune response enhancing compound chosen from the group of AMP-activated protein kinase (AMP-kinase) agonists.

19. A composition according to claim 18 wherein said AMP-kinase agonist is an AMP mimetic or adenosine pro-drug or derivative thereof which stimulates AMP-kinase.

20. A composition according to claim 18 or 19, wherein said immune response enhancing compound is selected from the group consisting of AICA-riboside (5-aminoimidazole-4-carboxamide riboside), SAICAR (5-amino-4-imidazole-N-succinocarboxamide riboside), ZMP (5-aminoimidazole-4-carboxamide-1-beta-D-ribofuranosyl-5′-monophosphate), 6 MPR (6-mercaptopurine riboside), AMP analogues and derivatives of any of said compounds.

21. A composition according to any of claims 18 to 20, wherein said immune response enhancing compound is AICA-riboside.

22. A composition according to any of claims 18 to 20, wherein said immune response enhancing compound is 6-MPR (6-mercaptopurine riboside).

23. A composition according to any of claims 18 to 22, wherein said immune response eliciting compound is a bacterial, viral, vegetable or parasitical antigen.

24. A composition according to any of claims 18 to 22, wherein said immune response eliciting compound is an allergen.

25. A composition according to any of claims 18 to 22, wherein said immune response eliciting compound is a live or attenuated microorganism.

26. Product containing an immune response eliciting molecule and an immune response enhancing compound chosen from the group of AMP-activated protein kinase (AMP-kinase) agonists or adenosine pro-drugs or derivates thereof which stimulate AMP-kinase, as a combined preparation for mixed or separate or sequential use in immuno-enhancing therapies.

27. Product according to claim 26, wherein said AMP-kinase agonist or adenosine pro-drug or derivate thereof which stimulates AMP-kinase is selected from the group consisting of AICA-riboside (5-aminoimidazole-4-carboxamide riboside), SAICAR (5-amino-4-imidazole-N-succinocarboxamide riboside), ZMP (5-aminoimidazole-4-carboxamide-1-beta-D-ribofuranosyl-5′-monophosphate), 6-MPR (6-mercaptopurine riboside), AMP analogues, and derivatives of any of said compounds.

28. Product according to claim 27, wherein said AMP-kinase agonist is AICA-riboside.

29. Product according to claim 27, wherein said AMP-kinase agonist is 6-MPR (6-mercaptopurine riboside).

30. Use of AICA-riboside as an adjuvant for a vaccine.

31. Use of 6-MPR (6-mercaptopurine riboside) as an adjuvant for a vaccine.
